(19) United States
(12) Patent Application Publication
(10) Pub. No.: US 2017/0044623 A1
(43) Pub. Date: Feb. 16, 2017

(54) NEW BIOMARKER FOR AML

(71) Applicants: INSERM (INSTITUT NATIONAL DE LA SANTÉ ET DE LA RECHERCHE MÉDICALE, Paris (FR); INSTITUT JEAN PAOLI & IRENE CALMETTES, Marseille (FR); CENTRE NATONAL DE LA RECHERCHE SCIENTIFIQUE(CNRS), Paris (FR); UNIVERSITÉ D'AIX MARSEILLE, Marseille 7 (FR)

(72) Inventors: Estelle DUPREZ, Marseille (FR); Boris CALMELS, Marseille (FR); Norbert VEY, Marseille (FR); Guillaume TIBERI, Garrigues (FR)

(21) Appl. No.: 15/305,137

(22) PCT Filed: May 7, 2015
(86) PCT No.: PCT/EP2015/060081

§ 371 Date: Oct. 19, 2016

(30) Foreign Application Priority Data
May 7, 2014 (EP) 14305672.9

(51) Int. Cl. C12Q 1/68

(52) U.S. Cl. CPC C12Q 1/6886; C12Q 2600/118; C12Q 2600/154; C12Q 2600/158

(57) ABSTRACT

The present invention relates to an in vitro method for predicting the survival time of a subject suffering from acute myeloid leukemia (AML) comprising determining, in a biological sample from the subject, the epigenetic profile of the H3K27.

### FIELD OF THE INVENTION

The invention relates to an in vitro method for predicting the survival time of a subject suffering from acute myeloid leukemia (AML) comprising determining, in a biological sample from the subject, the epigenetic profile of the H3K27.

### BACKGROUND OF THE INVENTION

AML is characterized by the clonal expansion of hematopoietic progenitors that have acquired numerous genetic and epigenetic alterations. In a substantial proportion of cases (˜40%), the leukemic cells lack any visible chromosomal abnormality (CN-AML); this group is highly heterogeneous in terms of biology and clinical outcome, which remains poorly understood [Mrozek K et al., 2007] The discovery of mutational events that affect genes involved in the regulation of hematopoietic commitment/differentiation, cell cycle and more recently epigenetics, has helped to dissect the molecular pathogenesis and enhance the classification of CN-AML [Dohner H. et al., 2010 and Miller C. A. et al., 2013]. Indeed, molecular profiling using targeted sequencing approaches has been shown to provide independent prognostic information, of potential value to guide treatment approach [Patel, J. P. et al., 2012].

Epigenetic changes in AML have been extensively studied, first looking at specific oncogenic or tumor suppressor loci, then more recently at the genome wide level, where DNA methylation profiling was used to identify potential biomarkers [Figueroa M. E. et al., 2010 and Deneberg S. et al., 2011].

However, no relevant biomarkers have been detected. Thus, there is still a need of new biomarkers and thus new methods of prognosis to better identify poor prognosis patients in AML.

### SUMMARY OF THE INVENTION

The inventors analyzed the epigenetic profile (methylation profile of the histones) of the HIST1 cluster located on 6p22.2 (26216000-2628500) and more precisely the methylation profile of the lysine 27 of the histone H3 (H3K27) in an enlarged cohort of 51 cases of CN-AML. This analyze revealed the presence of an abnormal epigenetic profile in about 50% of the patients characterized by high tri-methylation of H3K27 (H3K27me3) enrichment at the HIST1 cluster. This was associated with low expression of specific HIST1 genes, strongly associated with the presence of NPM1 mutation and significantly impacted on prognosis.

Thus, the invention relates to an in vitro method for predicting the survival time of a subject suffering from acute myeloid leukemia (AML) comprising determining, in a biological sample from the subject, the epigenetic profile of the H3K27.

### DETAILED DESCRIPTION OF THE INVENTION

A first object of the invention relates to an in vitro method for predicting the survival time of a subject suffering from acute myeloid leukemia (AML) comprising determining, in a biological sample from the subject the epigenetic profile of the H3K27.

As used herein, the term “epigenetic profile of the H3K27” denotes all modifications of the H3K27 located on the HIST1 cluster located on 6p22.2 (26216000-2628500) other than modification in the DNA sequence. Modifications which can affect the epigenetic profile of the lysine 27 of the histone H3 can be methylation (mono, bi or tri for example. Particularly, tri-methylation of the lys27 of the histone H3 are determined.

Thus, the invention also relates to an in vitro method for predicting the survival time of a subject suffering from acute myeloid leukemia (AML) comprising determining, in a biological sample from the subject the epigenetic profile of the H3K27 at the HIST1 cluster located on 6p22.2.

In another embodiment, the invention relates to an in vitro method for predicting the survival time of a subject suffering from acute myeloid leukemia (AML) comprising determining, in a biological sample from the subject, the epigenetic profile of the of the H3K27 at the HIST1 cluster located on 6p22.2 at position 26216000-2628500.

Thus, the invention relates to an in vitro method for predicting the survival time of a subject suffering from acute myeloid leukemia (AML) comprising i) determining in a sample obtained from the subject the histone methylation profile level of H3K27 ii) comparing the histone methylation profile level of H3K27 at step i) with its predetermined reference value and iii) providing a good prognosis when the histone methylation profile level determined at step i) is higher than its predetermined reference value, or providing a bad prognosis when the histone methylation profile level determined at step i) is lower than its predetermined reference value.

According to the invention, the acute myeloid leukemia (AML) can be an acute myeloid leukemia with normal karyotype (CN-AML), an acute myeloid leukemia with trisomy 8 and and acute leukemia with t(15;17).

According to the invention the subject can be treated with anti-AML compound like demethylating agent or by allograft.

As used herein the term “allograft” denotes a patient who has been treated by hematopoietic stem cell transplantation (HSCT). According to the term allograft, hematopoietic stem cells come from a donor related or not to the recipient but of the same species.

In another embodiment, methods according to the invention may be useful for predicting the overall survival (OS) of a patient suffering from acute myeloid leukemia (AML) or for predicting the free survival (FS) of a patient suffering from acute myeloid leukemia (AML).

In a particular embodiment, the invention relates to a method for predicting the overall survival (OS) of a patient suffering from acute myeloid leukemia (AML) comprising i) determining in a sample obtained from the subject the histone methylation profile level of H3K27 ii) comparing the histone methylation profile level of H3K27 at step i) with its predetermined reference value and iii) providing a good prognosis when the histone methylation profile level determined at step i) is higher than its predetermined reference value, or providing a bad prognosis when the histone methylation profile level determined at step i) is lower than its predetermined reference value.

In a particular embodiment, the invention, the invention relates to a method for predicting the free survival (FS) of a patient suffering from acute myeloid leukemia (AML) comprising i) determining in a sample obtained from the subject the histone methylation profile level of H3K27 ii) comparing the histone methylation profile level of H3K27 at step i) with its predetermined reference value and iii) providing a good prognosis when the histone methylation profile level determined at step i) is higher than its predetermined reference value, or providing a bad prognosis when the histone methylation profile level determined at step i) is lower than its predetermined reference value.

As used herein, the term “Overall survival (OS)” denotes the percentage of people in a study or treatment group who are still alive for a certain period of time after they were diagnosed with or started treatment for a disease, such as AML (according to the invention). The overall survival rate is often stated as a five-year survival rate, which is the percentage of people in a study or treatment group who are alive five years after their diagnosis or the start of treatment.

As used herein, the term “Free Survival (FS)” (or Event-Free-Survival) denotes the length of time after primary treatment for a cancer ends that the patient remains free of certain complications or events that the treatment was intended to prevent or delay. These events may include the return of the cancer or the onset of certain symptoms, such as bone pain from cancer that has spread to the bone.

As used herein, the term “histone methylation profile level of H3K27” denotes the level of methylation of the Histone H3 on the lysine 27 in the HIST1 cluster located on 6p22.2 (26216000-2628500) that is to say the number of CH3 group on the Histone H3 on the lysine 27.

According to the invention, the histone methylation on H3K27 can be a mono-methylation, di-methylation or a tri-methylation.

In a particular embodiment, the H3K27 is tri-methylated.

Thus, the invention also relates to an in vitro method according to claim 1 comprising i) determining in a sample obtained from the subject the histone tri-methylation profile level of H3K27 ii) comparing the histone tri-methylation profile level of H3K27 at step i) with its predetermined reference value and iii) providing a good prognosis when the histone tri-methylation profile level determined at step i) is higher than its predetermined reference value, or providing a bad prognosis when the histone tri-methylation profile level determined at step i) is lower than its predetermined reference value.

As used herein the term “subject” refers to a human or animal, including all vertebrates, e.g., mammals, such as primates (particularly higher primates), sheep, dog, rodents (e.g., mouse or rat), guinea pig, goat, pig, cat, rabbit, cow; and non-mammals, such as chicken, amphibians, reptiles, etc. In a preferred embodiment, the subject is a human. In another embodiment, the subject is an experimental animal or animal suitable as a disease model.

As used herein the term “biological sample” in the context of the present invention is a biological sample isolated from a subject and can include, by way of example and not limitation, bodily fluids and/or tissue extracts such as homogenates or solubilized tissue obtained from a subject. Tissue extracts are obtained routinely from tissue biopsy and autopsy material. Bodily fluids useful in the present invention include blood, bone marrow aspirate, urine, saliva or any other bodily secretion or derivative thereof. As used herein “blood” includes whole blood, plasma, serum, circulating cells, constituents, or any derivative of blood. In a particular embodiment, the biological sample is a blood sample, more particularly a biological sample comprising circulating white blood cells (WBC).

Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample”.

In a particular embodiment, the sample includes nucleic acids.

Methods for extracting chromatin from biological samples and determining the histone methylation level are well known in the art. Commonly, chromatin isolation procedures comprise lysis of cells after one step of crosslink that will fix proteins that are associated with DNA. After cell lysis, Chromatin is fragmented, immunoprecipitated and DNA is recovered. DNA is then extracted with phenol, precipitated in alcohol, and dissolved in an aqueous solution.

The H3K27 methylation level can be determined by chromatin IP (see for example Boukarabila H., et al, 2009) ChIP-chip or by ChIP-qPCR (see for example the materiel and methods part and Wu J. et al., 2006).

According to the invention the “reference value” is the histone methylation level of H3K27 in the HIST1 cluster determined in a biological sample of a subject not afflicted by an AML. Preferably, said normal level of histone methylation is assessed in a control sample (e.g., sample from a healthy patient, which is not afflicted by an AML) and preferably, the average e histone methylation profile level of said gene in several control samples.

According to the invention, the “reference value” or “cut off value” is determined by considering the distribution of the 5 HIST1 median values for all patients. This clearly shows a bimodal distribution of the patients. The first group that has a very homogeneous low median value below 10 in comparison to the second that has median values above 10 (see materiel and methods, part “ChIP-qPCR normalization” of the patent application).

In a further embodiment of the invention, methods of the invention comprise measuring the histone methylation profile level of at least one further biomarker or prognostic score.

The term “biomarker”, as used herein, refers generally to a cytogenetic marker, a molecule, the expression of which in a sample from a patient can be detected by standard methods in the art (as well as those disclosed herein), and is predictive or denotes a condition of the subject from which it was obtained.

Various validated prognostic biomarkers or prognostic scores may be combined to the H3K27me3 in order to improve methods of the invention and especially some parameters such as the specificity (see for example Comelissen et al. 2012).

For example, the other biomarkers may be selected from the group of AML biomarkers consisting of cytogenetics markers (like t(8;21), t(15;17), inv(16), t(16;16), t(9;11), -5, -7, 5q-, 7q-, 11q23, excl. t(9;11), Inv(3), t(3;3), t(6;9), t(9;22) see for example Grimwade et al., 2010 or Byrd et al., 2002), lactate dehydrogenase (see for example Haferlach et al 2003), FLT3, NPM1, CEBPα (see for example Schnittger et al., 2002).

The prognostic scores that may be combined to HIST1 H3K27me3 may be for example the Hematopoietic Cell Transplantation Comorbidity Index (HCT-CI) (Sorror et al 2005), the comorbidity and disease status (Sorror et al 2007) or the disease risk index (DRI) (Armand et al 2012).

According to the invention, detection of a mutation in the gene NPM1 can be added to the determination of the histone methylation profile level of H31(27 for predicting the survival time of a subject suffering from acute myeloid leukemia (AML).

As used herein, the term “NPM1” denotes a gene coding fort the protein nucleophosmin (NPM), also known as nucleolar phosphoprotein B23 or numatrin. The protein NPM1 is associated with nucleolar ribonucleoprotein structures and bind single-stranded and double-stranded nucleic acids, but it binds preferentially G-Quadruplex forming nucleic acids. NPM1 mutations are known to be biomarkers for AML (Falini B et al., 2009).

Thus, the invention also relates to an in vitro method for predicting the survival time of a subject suffering from acute myeloid leukemia (AML) comprising determining, in a biological sample from the subject the epigenetic profile of the H3K27and if a mutation in the gene NPM1 is present.

Thus, the invention relates to an in vitro method for predicting the survival time of a subject suffering from acute myeloid leukemia (AML) comprising i) determining in a sample obtained from the subject the histone methylation profile level of H3K27 and the presence of NPM1 mutations ii) comparing the histone methylation profile level of H3K27 at step i) with its predetermined reference value and iii) providing a good prognosis when the histone methylation profile level determined at step i) is higher than its predetermined reference value and when there is a mutation in NPM1, providing a good prognosis when the histone methylation profile level determined at step i) is higher than its predetermined reference value and when there is no mutation in NPM1 and providing a bad prognosis when the histone methylation profile level determined at step i) is lower than its predetermined reference value and when there is a mutation in NPM11 or where there is no mutation in NPM1.

According to the invention, determination of the level expression for genes ofthe HIST1 cluster can be added to the determination of the histone methylation profile level of H3K27 for predicting the survival time of a subject suffering from acute myeloid leukemia (AML).

According to the invention, the genes of the HIST1 cluster can be HIST1H2BG, HIST1H2AE, HIST1H3E, HIST1H1D, HIST1H4F, HIST1H4G, HIST1H3F, HIST1H2BH, HIST1H3G, HIST1H2BI or HIST1H4H.

Accession numbers of the different genes are: HIST1H2BG: Ref Seq NM_003518.3 GenBank: M60750.1; HIST1H2AE: Ref Seq NM_021052 GenBank: M60752; HIST1H3E: Ref Seq NM_003532 GenBank: M60746; HIST1H1D: Ref Seq NM_005320 GeneBank: M60747; HIST1H4F: Ref Seq NM_003540 GeneBank: M60749; HIST1H4G: Ref Seq NM_003547 GeneBank: Z80788; HIST1H3F: Ref Seq NM_021018 GeneBank: Z80786; HIST1H2BH: Ref Seq NM_003524 GeneBank: Z80781; HIST1H3G: Ref Seq NM_003534 GeneBank: Z80785 and HIST1H2BI: Ref Seq NM_003525 GeneBank: Z80782.

Thus, the invention also relates to an in vitro method for predicting the survival time of a subject suffering from acute myeloid leukemia (AML) comprising determining, in a biological sample from the subject, the epigenetic profile of the H3K27 and the expression level of at least one gene selected in the group consisting of HIST1H2BG, HIST1H2AE, HIST1H3E, HIST1H1D, HIST1H4F, HIST1H4G, HIST1H3F, HIST1H2BH, HIST1H3G, HIST1H2BI or HIST1H4H.

Thus, the invention also relates to an in vitro method for predicting the survival time of a subject suffering from acute myeloid leukemia (AML) comprising i) determining in a sample obtained from the subject the histone methylation profile level of H3K27 and the expression level of at least one gene selected in the group consisting of HIST1H2BG, HIST1H2AE, HIST1H3E, HIST1H1D, HIST1H4F, HIST1H4G, HIST1H3F, HIST1H2BH, HIST1H3G, HIST1H2BI or HIST1H4H ii) comparing the histone methylation profile level of H3K27 and the expression level of the gene at step i) with their predetermined reference value and iii) providing a good prognosis when the histone methylation profile level determined at step i) is higher than its predetermined reference value and when the expression level of at least one gene is lower than it predetermined value, providing a good prognosis when the expression level determined at step i) is higher than its predetermined reference value and when the expression level of at least one gene is lower than it predetermined value, or providing a bad prognosis when the expression level determined at step i) is lower than its predetermined reference value and when the expression level of at least one gene is higher or lower than its predetermined value.

Methods for determining the expression level of genes are well known in the art. For example, PCR, rtPCR or sequencing can be used.

The present invention also relates to kits for predicting the survival time of a subject suffering from acute myeloid leukemia (AML) comprising means for determining, in a biological sample from the subject the epigenetic profile of the H3K27.

In a particular embodiment, the invention relates to kits for predicting the survival time of a subject suffering from acute myeloid leukemia (AML) comprising means for determining, in a biological sample from the subject the histone methylation profile level of H3K27.

The invention also relates to the use of the epigenetic profile of the H3K27 as a biomarker for the survival time of a subject suffering from acute myeloid leukemia (AML).

The invention also relates to the use of the histone methylation profile level of H3K27 as a bio marker for the survival time of a subject suffering from acute myeloid leukemia (AML).

A method of treatment of an AML in a subject in need thereof comprising the step of:

1. determining the epigenetic profile of the H31(27 according to the invention and;

2. administrating to said subject a compound useful for the treatment of AML when the prognosis of the subject is bad as determined by methods of the invention.

Compound useful for the treatment of AML are well known in the art (see for example Sweet K. et al., 2014).

The treatment used can be an allograft (allogeneic stem cell transplantation) or all compound used to treat AML like Anthracycline and cytarabine “3+7” combination), and others chemotherapies.

The invention will be further illustrated by the following figures and examples. However, these examples and figures should not be interpreted in any way as limiting the scope of the present invention.

### FIGURES

FIG. 1: a gain of H3K27me3 at the HIST1 cluster distinguishes two sub-groups of CN-AML patients. (A) Analyses of H3K27me3 levels by ChIP-qPCR in 51 CN-AML, three normal bone marrow and three CD34+ sorted cord blood samples, at 5 HIST1 regions. Enrichment was calculated as percentage of bound/input and normalized with HOXD4 and GAPDH. Data are presented as heatmaps. Each column represents a patient sample, sorted by upward median values for HIST1 enrichment. (B) Comparison of expression level of four histone genes between high H3K27me3-enriched and low H3K27me3-enriched patients. Statistical significance was estimated using Mann Whitney t test, (***) p≦0.0001. High refers to the high H3K27me3 level patients, low refers to the low H3K27me3 level patients, nLC stands for non-leukemic cells and refers to normal bone marrow cells or CD34+ sorted cord blood cells.

FIG. 2: High H3K27me3 HIST1 level associates with NPM1 mutation but is a stronger outcome predictor.

(A) Comparison of H3K27me3 level of 5 histone promoter regions between NPM1mut (n=46) and NPM1wt (n=25). (B) Allo-censored leukemia free survival. Statistical significance was estimated using the long rank test.

TABLE 1
Clinical, mutational and patient characteristics were analyzed according to
HIST1 H3K27me3 level in CN-AML patients.

| | | | HIST1 H3K27me3 | | | | |
|---|---|---|---|---|---|---|---|
| | All patients | | Low | | High | | |
| | n = 86 | | n = 42 | | n = 44 | | pvalue |
| Gender | F: 42 | (49%) | F: 19 | (45.2%) | F: 23 | (52.3%) | 0.527 |
| | M: 44 | (51%) | M: 23 | (54.8%) | M: 21 | (47.7%) | |
| Age at diag. | 56.06 | | 53.92 | | 58.10 | | 0.164 |
| Age at death | 59.79 | | 58.27 | | 61.36 | | 0.354 |
| Mean WBC | 82 | (10-641) | 77.33 | | 86.68 | | 0.929 |
| Complete Response | 73 | (84.88%) | 33 | (78.57%) | 40 | (90.91%) | 0.139 |
| Allo | 20 on 73 | (27.30%) | 9 on 33 | (27.27%) | 11 on 40 | (27.50%) | 1.000 |

| | | | | |
|---|---|---|---|---|
| OS (months) | 16.26 | 15.21 | 23.11 | 0.167 |
| LFS Allo (months) | 11.97 | 8.92 | 13.33 | 0.0053 |
| FAB | | | | |
| M0 | 1 | 1 | 0 | |
| M1 | 39 | 18 | 21 | |
| M2 | 5 | 2 | 3 | 0.940 |
| M4 | 15 | 8 | 7 | |
| M5 | 24 | 13 | 11 | |
| NR | 1 | 0 | 1 | |

Mutations

| | | | | | | | |
|---|---|---|---|---|---|---|---|
| NPM1 | 46 on 71 | (64.79%) | 14 on 35 | (40.00%) | 32 on 36 | (88.90%) | 1.75E−005 |
| FLT3-ITD | 36 on 81 | (44.44%) | 16 on 41 | (39.00%) | 20 on 40 | (50.00%) | 0.375 |
| FLT3-TKD | 8 on 50 | (16%) | 5 on 28 | (18.90%) | 3 on 22 | (13.40%) | 1.000 |
| DNMT3A-R882 | 15 on 50 | (30%) | 9 on 28 | (32.10%) | 6 on 22 | (27.30%) | 0.765 |
| DNMT3A-other | 8 on 50 | (16%) | 3 on 28 | (10.70%) | 5 on 22 | (22.70%) | 0.277 |
| TET2 | 12 on 50 | (24%) | 4 on 28 | (14.30%) | 8 on 22 | (36.40%) | 0.099 |
| IDH2 R140 | 9 on 50 | (18%) | 3 on 28 | (10.70%) | 6 on 22 | (27.30%) | 0.157 |
| IDH1 R132 | 3 on 50 | (6%) | 2 on 28 | (7.10%) | 1 on 22 | (4.50%) | 1.000 |
| ASLX1 | 4 on 50 | (8%) | 3 on 28 | (10.70%) | 1 on 22 | (4.50%) | 3.621 |
| WT1 | 6 on 50 | (12%) | 6 on 28 | (21.40%) | 0 on 22 | (0%) | 0.028 |

TABLE 2
multivariate analysis for allo-censored LFS. Multivariate analysis
for HIST1 H3K27me3 level on LFS-allo. OR indicates odds ratio;
HR, hazard ratio; CI, confidence interval. Variables considered are
HIST1 H3K27me3 level (low vs. high), age at diagnosis (<56)
NPM1mutation (absent vs. present), FLT3-ITD (absent vs. present).
Multivariate analysis LFS-allo

| Variable | Hazard Ration | | pvalue |
|---|---|---|---|
| HIST H3K27 me3 high | 0.302 | (0.14, 0.64) | 0.0019 |
| Age at diagnosis | 2.033 | (1.21, 4.49) | 0.0114 |
| NPM1mut | 1.77 | (0.86, 3.67) | 0.1220 |
| FLT3-ITD | 1.35 | (0.71, 2.56) | 0.3585 |

### EXAMPLE

### Material & Methods

Patient Samples.

We selected cryopreserved primary bone marrow or peripheral blood samples that were obtained at diagnosis from 86 de novo CN-AML patients admitted at Institut Paoli-Calmettes (IPC). Blast cells are routinely separated from blood or marrow samples through density-gradient (ficoll) separation, and stored in liquid nitrogen. Only CN-AML samples containing more than 80% of blasts were selected from the IPC Biological Resources Center inventory for the purpose of genetic and epigenetic studies. Informed consent was available for all patients, and the study was approved by our institutional review board (COS).

Cytogenetic analysis using conventional techniques was performed as part ofthe routine diagnostic work-up. All patients were treated according to national AML guidelines. They received standard induction regimens (i.e. Anthracycline and cytarabine “3+7”combination), and were evaluated for response to induction chemotherapy. Patients who achieved a complete remission received one or two cycles of consolidation therapy (intermediate to high-dose cytarabine) and/or allogeneic stem cell transplantation. Clinical characteristics of patients are shown in table 1.

For mutation screening, genomic DNA was extracted using standard protocol. Mutation status of FLT3, NPM1, DNMT3A, TET2, IDH1, IDH2, ASXL1 and WT1 was established by high-throughput sequencing using HaloPlex Target Enrichment (Agilent Technologies, Santa Clara, Calif.) on an Illumina HiSeq 2000 platform (Illumina, San Diego, Calif.).

ChIP-chip and ChIP Followed by qPCR Experiments (ChIP-qPCR).

Patient samples were thawed and viability estimated by trypan blue. Five million cells were used for ChIP-chip experiments and one million for ChIP-qPCR. Chromatin IPs were performed as previously described (Boukarabila, H. et al., 2009). Two different anti-H3K27me3 antibodies were used (#07-449; Millipore or #ab6002; Abcam). For ChIP-chip analyses (n=35), input and ChIPed DNA samples were labeled with Cy3-dUTP and Cy5-dUTP (Perkin-Elmer, Mass., USA), respectively. Hybridization was performed onto custom promoter oligonucleotide (ChIP/CH3 2×400 k SurePrint G3 personnal; Agilent Technologies27) arrays containing more than 400,000 independent genomic oligonucleotides covering 6 kb of promoter regions (−3 to +3 kb, with respect to the transcription start site) of refseq genes (UCSC HG18). Images were scanned using a DNA microarray scanner and processed using the Agilent Feature Extraction Software version 9.5.1 (Agilent Technologies, Santa Clara Calif., USA). For ChIP-qPCR (n=54), DNA was purified on Chelex, cleaned up on Qiaquick (Qiagen, Venlo, Netherlands) and analyzed by qPCR using power SYBR Green on a 7500 Real time PCR system (Applied Biosystem, Foster City, Calif., USA). IgG control “cycle over the threshold” Ct values were subtracted from Input or IP Ct values, and converted into bound value by 2(-(IP Ct or input Ct-IgG IP Ct). For the ChIP-qPCR, patient samples were only considered when passing our quality control tests that were (1) highly enriched for H3K27me3 at the HOXD4 locus (positive control) comparatively to the GAPDH locus (negative control); (2) possibility of gender determination by looking at H3K27me3 levels on the GEMIN8 promoter located on the X-chromosome. Primers sequences will be provided upon request.

Expression Analysis.

Total RNA was extracted using the RNeasy Mini kit according to the manufacturer's protocol (Qiagen), Prior to cDNA synthesis, RNA samples were treated with DNaseI (Sigma-Aldrich, Saint-Louis, Mo., USA). Reverse transcription was performed with 100 ng of the total RNA, oligodT, dNTPs, DTT, buffer and the high fidelity retrotranscriptase. cDNA was analyzed by qPCR using power SYBR Green on a 7500 Real time PCR system (Applied Biosystems). Primers sequences will be provided upon request.

Bio Informatics

ChIP-chip Analyses

For each patient sample, normalized log2 enrichment ratios (ChIP/Input) were calculated using CoCAS software 28. Each promoter (corresponding to 6 kb region) was analyzed as three independent regions of 2 kb length. (reg1: −3 −1 kb; reg2: −1 +1 kb; reg3: +1 +3 kb). Region value was calculated by averaging the normalized intensity of the probes with custom scripts developed using the Perl programming language. 62,979 regions were initially identified and reduced to 48,690 unique regions after removal of repetitive regions of the genome. Hyper variant regions between patients (standard deviation>0.6) were selected after removing regions with negative values for all patients and regions located on the sex chromosomes. These filters defined a shorted list of 586 variant promoter regions. For unsupervised hierarchical clustering and heatmap generation Average Complete Uncentered Pearson Coefficient distance was calculated using custom scripts based on the Amap (R package version 0.8-7. http://CRAN.R-project.org/package=amap) and Heatplus Bioconductor packages. Since one of the X-chromosomes is epigenetically silenced with a mechanism involving H3K27me3 repression 29 we validated our data analysis pipeline by determining patient gender with promoter regions on the X chromosome (data not shown). A bootstrap step was performed for the detection of statistically significant gene clusters using R package pvclust (10,000 iterations) 30. Only clusters with an Approximately Unbiased (AU) corrected p value above 0.95 and containing a minimum of 5 unique promoters were selected. Heatmaps, plots of the large variance components and defined clusters were used to visualize the results (data not shown).

ChIP-qPCR Normalization

First ChIP-qPCR values Xᵢ were normalized relative to xGAPDH (our negative control value) and log2 transformed.

x′ᵢ=log2(xᵢ/xGAPDH)

Then, X′ᵢ were normalized relative to x′HOXD4 (our positive control value)

x″ᵢ=x′ᵢ/x′ HOXD4

To set our negative control (GAPDH) at 1% and our positive control (HOXD4) to 100% we applied the formula: x″′ᵢ=2^{(x″I×log}2(100))

Samples with a x″′ᵢ lower than 10 were considered H3K27me3 low, samples with a X″′ᵢ greater than 10 were considered H3K27me3 high and samples with x″′ greater than 150 were excluded from the study.

Gene Ontology Enrichment

Gene Ontology term enrichment was measured by hypergeometric distribution using custom scripts.

Statistical Analyses

Patients having high and low HIST1H H3K27me3 levels were compared for clinical and molecular features using the Gehan-Wilcoxon and khi2 tests available in R package “survival” (R package version 2.37-4, http://CRAN.R-project.org/package=survival). In our analysis, only patients that presented Complete Remission (CR) were selected. Overall survival (OS) was defined as the time between date of RC and date of latest news (death or end of clinical follow-up). LFS is defined as the period between the RC and the date of relapse, death or latest news. For deceased patients, in case of relapse, the end of the survival time is the relapse date. For the others, the end of the survival time is the date of death event and allo-transplanted patients were censored at the transplantation date (LFS-allo). Estimated probabilities of OS and LFS-allo were calculated using the Kaplan-Meier method and the log rank test was used to evaluate differences between survival distributions.

### Results

Here we used epigenetic profiling to gain further insights into CN-AML, which is much more poorly characterized compared to rarer subsets of AML with chimeric oncoproteins consequent upon balanced chromosomal rearrangements. As EZH2 is the histone H3 methyl transferase catalytic subunit of Polycomb group (PcG) complexes that has been most frequently implicated in the pathogenesis of human malignancies10 but very rarely found to be mutated in AML (<1%)11, we analyzed global H3K27me3 profiles by ChIP coupled with hybridization on oligonucleotide promoter arrays (Chip-chip) in 35 CN-AML patient samples. By doing so, we could demonstrate variations in EZH2 activity across CN-AML, the variation was mostly observed in promoter regions of genes involved in cell surface and chromatin organization (data not shown) and we observed a significant over-representation of PcG target genes being differentially H3K27 tri-methylated (data not shown), including 10 genes with known impact in AML pathogenesis. The observed variation in EZH2 activity supports the hypothesis of site-specific deregulation of PcG/EZH2 target genes in AML12.

Unsupervised hierarchical clustering analysis (HCL) of these highly H3K27me3 variant promoter regions revealed, among different genomic regions, 9 significant clusters with homogeneous enrichment levels (data not shown). Cluster #3 was noteworthy being a robust cluster (dendrogram scale between 0.6 and 1) comprised of 22 sequential genomic regions, all belonging to the HIST1 gene locus located on chromosome 6p21. Hierarchical clustering performed with H3K27me3 enrichment of the 22 HIST1 genomic regions clearly distinguished 2 groups of patients based on their H3K27me3 level: one group with high and homogeneous H3K27me3 levels, and the other with low H3K27me3 levels (data not shown). These distinct H3K27me3 patterns at the HIST1 cluster did not correlate with patient characteristics, such as age or gender (data not shown). Specificity of these differences in H3K27me3 at the HIST1 cluster, was analyzed by supervised clustering of H3K27me3 levels at the majority of HIST1 cluster promoter regions on chromosome 6 within our patient cohort (data not shown). The clustering showed that heterogeneity in H3K27me3 levels was restricted to the 22 regions of chromosome 6, as previously revealed by our HCL analysis, while the other HIST1 promoter regions were homogeneous across patient samples (data not shown). We have thus identified a clustered genomic region that harbors a dramatic difference in H3K27me3 level within our group of CN-AML patients, demonstrating that epigenetic deregulation can affect localized chromosomal regions in tumor cells lacking chimeric oncoproteins.

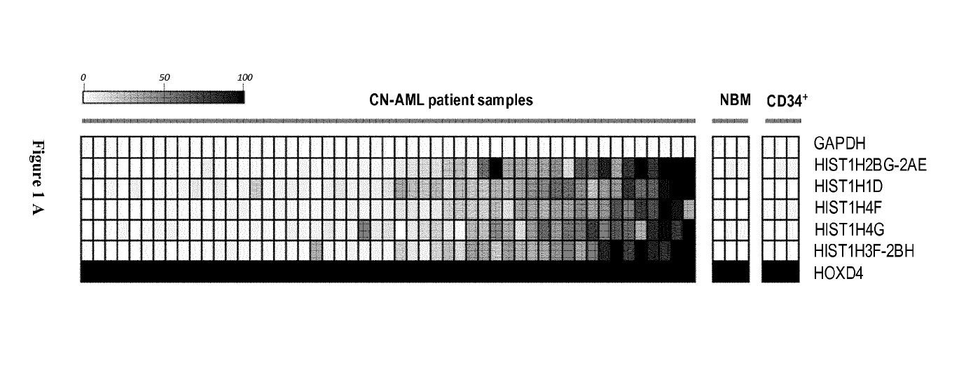

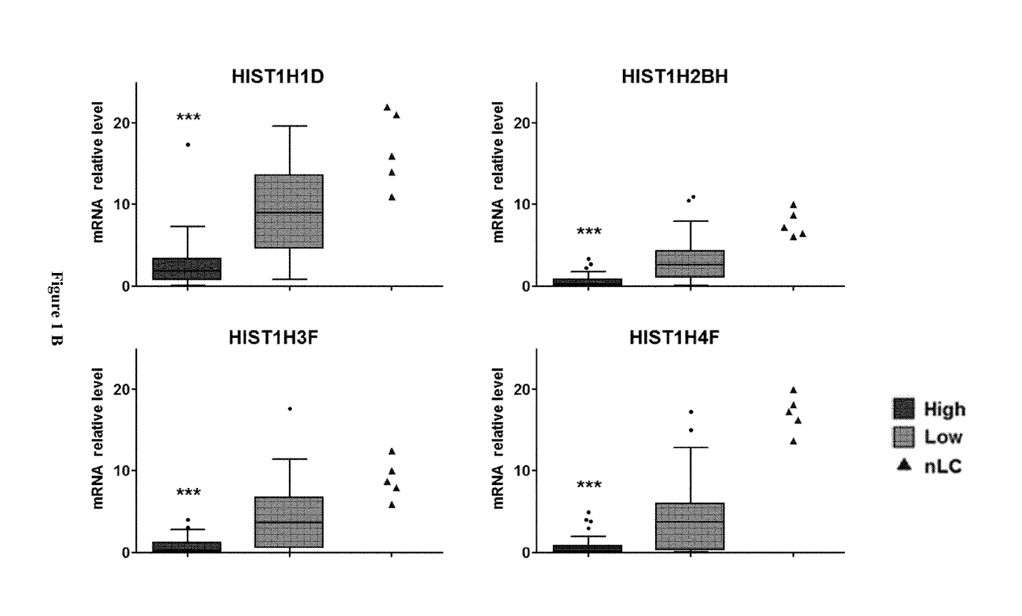

HIST1 H3K27me3 enrichment differences were independently confirmed by reanalyzing four patient samples using ChIP followed by RT-qPCR (ChIP-qPCR). We confirmed the difference in H3K27me3 enrichment at 5 “variable” HIST1 regions (HIST1H2BG-2AE, HIST1D, HIST1H4F, HIST 1H4G and HIST1H3F-2BH) but not in the two flanking regions HIST1H1E and HIST1H4E, in two “high H3K27me3” and in two “low H3K27me3” patients (data not shown). In order to independently extend this observation, we analyzed the H3K27me3 status by ChIP-qPCR, at the same five HIST1 genomic locations, in an independent cohort of 51 CN-AML patients. These experiments confirmed the previously described clustered H3K27me3 profile at the HIST1 locus, distinguishing two separate groups of patients (FIG. 1A). H3K27me3 ChIP experiments using non-leukemic hematopoietic cells (normal bone marrow, CD34+sorted cord blood) revealed that HIST1 cluster promoters are not normally enriched for H3K27me3 (FIG. 1A). This observation was confirmed by the analysis of publicly available ChIP-seq data (GEO, GSM773041) that shows absence of the H3K27me3 mark at the HIST1 locus in human CD34+ hematopoietic stem cells (data not shown). Together, these results highlight that gain of H3K27me3 at the HIST1 locus provides an epigenetic signature that discriminates two subgroups of CN-AML patients.

H3K27me3 is an epigenetic mark of repression that is associated with poor transcription rate13. In order to compare H3K27me3 status and HIST1 mRNA levels, we determined expression of four HIST1 genes (HIST1H1D, HIST1H2BH, HIST1H3F, HIST1H4F) by real time quantitative PCR, in the whole cohort of CN-AML samples (n=86). Expression of the 4 HIST1 genes was significantly higher in the “low H3K27me3 level” group, as compared to the “high H3K27me3 level” group (p value≦0.0001; FIG. 1B). In addition, we confirmed that non-leukemic hematopoietic cells express high levels of HIST1 gene mRNA. The inverse correlation observed between H3K27me3 levels and H1ST1 expression, suggests that the elevated level of H3K27me3 might be involved in the transcriptional repression of some of the HIST1 cluster genes in CN-AML patient blasts.

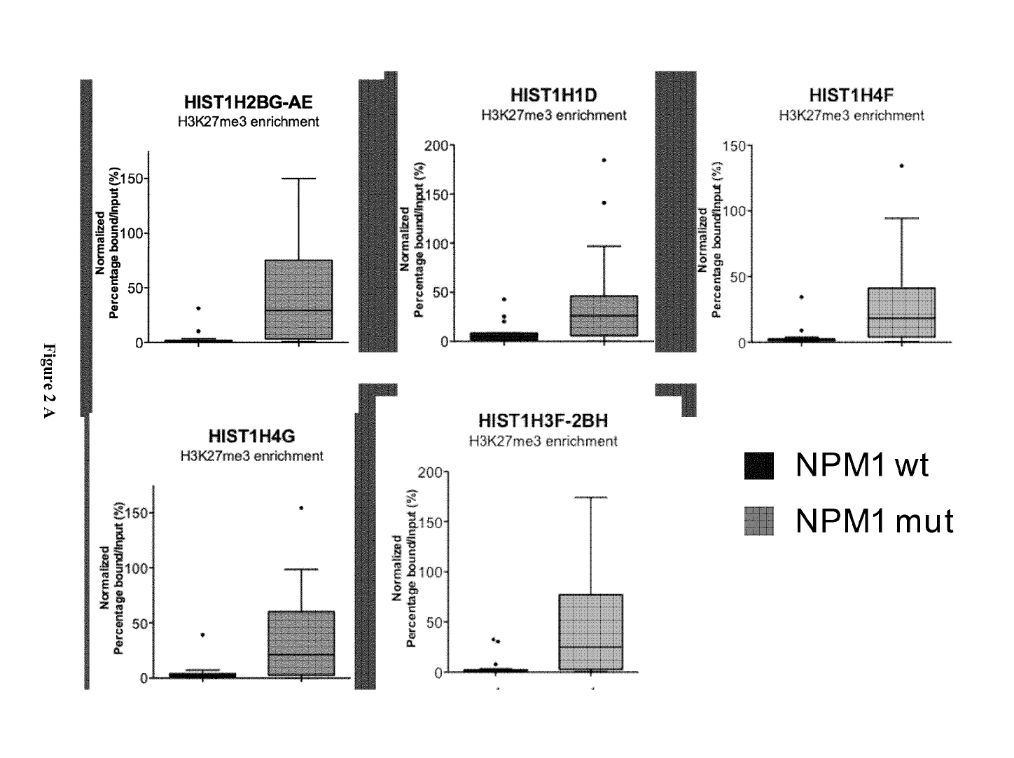

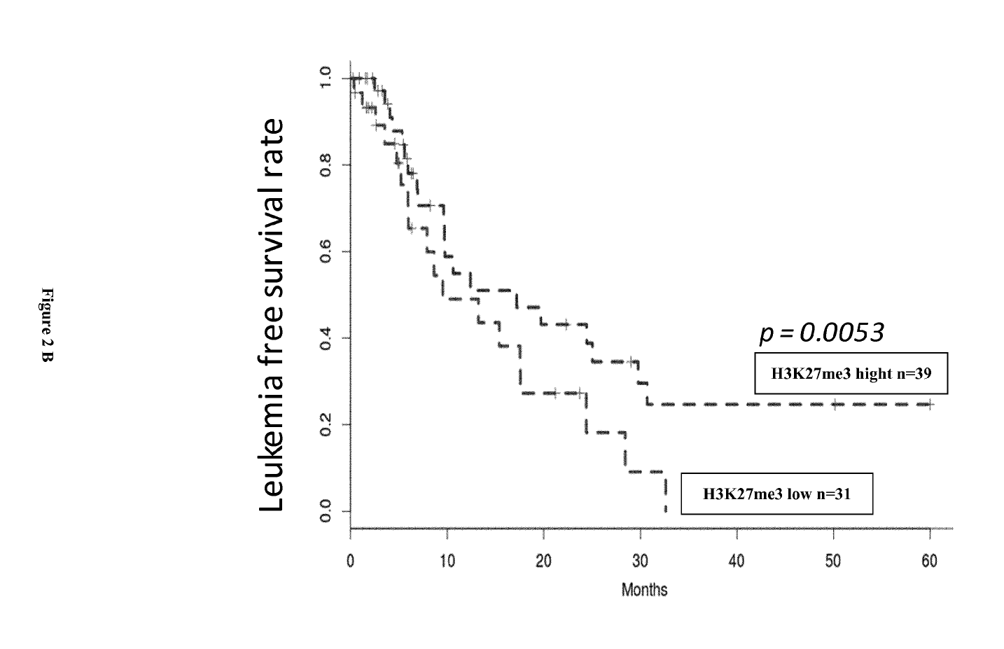

CN-AML samples were split in two groups, according to the median H3K27me3 enrichment levels at the HIST1 cluster genes. These two groups were analyzed for clinical and molecular characteristics: no significant differences in median age of AML at diagnosis were observed, suggesting that H3K27me3 enrichment does not correlate with aging (Table 1). In addition, the two groups were not associated with a specific French-American-British (FAB) class, suggesting that H3K27me3 level at the HIST1 locus is not biased by the differentiation stage of the leukemic cells (Table 1). Patients with high H3K27me3 level had a markedly higher incidence of NPM1 mutation (89% vs 40%; p=1.75×10-5) and a substantially lower incidence of WT1 mutation (0% vs 20%; p=0.028). No significant association was observed with FLT3 (ITD and TKD), IDH1/2, DNMT3A, nor ASXL1 mutations (Table 1). We compared H3K27me3 levels on 5 HIST1 promoter regions, in both NPM1-mutated and NPM1-wt (in total 86 AML cases). For each of the 5 promoter regions, we observed that the higher H3K27me3 levels were restricted to NPM1 mutated AML cases (all p values≦0.0001; FIG. 2A). These findings support the previous observations of low-level expression of some HIST1 genes in NPM1-mutated AML and provide new molecular insight to understand leukemic mechanism linked to NPM1 mutations.

Co-occurrence analysis reveals a favorable mutation profile (high incidence of NPM1 mutations and no mutations in WT1) associated with high H3K27me3 profile (data not shown), that was in accordance with the better outcome of the high H3K27me3 level sub-group of CN-AML patients. Patients with high HIST1 H3K27me3 level had a longer overall survival (23.11 vs 15.21 months; p=0.167) and significant longer leukemia free survival at 5 years when allo-grafted patients were censored (LFS-allo; 13.33 vs 8.92 months p=0.0053) (Table 1; FIG. 2B). Interestingly, the prognostic significance of HIST/ H3K27me3 was independent of the age and the presence of NPM1 or FLT3 mutations in multivariate analyses (Table 2), These data suggested that HIST1 H3K27me3 level is a biomarker that discriminates biologically distinct subsets of CN-AML with differing clinical outcome.

Here, we described a gain in H3K27me3 at a defined region of the HIST1 cluster as a new epigenetic alteration in CN-AML that affects the expression of canonical histone. Although mechanisms underlying this gain in H3K27me3 are unknown, it is interesting to note that this specific gain in H3K27me3 is not associated with alteration in pathways known to influence the activity of EZH2 (table 1). Recently, mutations in the histone variant H3.3 were described in pediatric cancer16,17, inducing a global loss of Lys27 methylation with a gain of Lys27 methylation in some genomic regions, suggesting a global H3K27me3 reshaping. The epigenetic signature that we have revealed here might be the result of an indirect deregulation of EZH2.

Noticeably, this specific HIST1 gene signature was not highlighted by genomic expression profile data, and our unpublished data on the same cohort. This could be explained by the intrinsic features of the histone genes: redundancy of the 5 canonical histone genes as well as subtle differences in their corresponding proteins that renders investigations on the function and expression of individual histone genes difficult. How deregulation of some canonical histone genes could contribute to leukemogenesis is unclear. Growing evidence suggests that mutations in histone genes are associated with hematological malignancies: mutations in HIST1H3B and HIST1H1C have been found in diffuse large B cell lymphomas (DLBCL). Interestingly, focal deletion of a histone gene cluster at 6p22, overlapping with our H3K27me3 region, has been described in 19% of near-haploid cases of acute lymphoblastic leukemia. In line with these findings, our data suggest a common neoplastic mechanism that may alter histone function and regulation through affecting nucleosome structure and function by altering histone-DNA interactions, chromatin compaction or interactions with other effectors that bind to histones.

In Conclusion, we have found that the H3K27me3 profile varies across CN-AML patients, revealing the complexity of epigenetic regulation in an otherwise homogeneous pathological entity. We identified two subgroups of CN-AML patients that differ according to H3K27me3 levels at genes within the HIST1 cluster. The enrichment of H3K27me3 at the HIST1 cluster is associated with NPM1 mutations and provides a new molecular marker with potential value in diagnosis and prognosis. This example supports the interest of using epigenetic profiling for identifying new deregulated loci linked to pathology.

### REFERENCES

Throughout this application, various references describe the state of the art to which this invention pertains. The disclosures of these references are hereby incorporated by reference into the present disclosure.

Cornelissen J J1, Gratwohl A, Schlenk R F, Sierra J, Bornhäuser M, Juliusson G, Råcil Z, Rowe J M, Russell N, Mohty M, Löwenberg B, Socié G, Niederwieser D, Ossenkoppele G J. The European LeukemiaNet AML Working Party consensus statement on allogeneic HSCT for patients with AML in remission: an integrated-risk adapted approach. Nat Rev Clin Oncol. 2012 October;9(10):579-90. doi: 10.1038/nrclinonc.2012.150. Epub 2012 Sep 4.

Falini B, Sportoletti P, Martelli M P. Acute myeloid leukemia with mutated NPM1: diagnosis, prognosis and therapeutic perspectives. Curr Opin Oncol. 2009 November;21(6):573-81.

Schnittger Susanne, Claudia Schoch, Martin Dugas, Wolfgang Kern, Peter Staib, Christian Wuchter, Helmut Löffler, Cristina Maria Sauerland, Hubert Serve, Thomas Bu\ddot{}chner, Torsten Haferlach, and Wolfgang Hiddemann. Analysis of FLT3 length mutations in 1003 patients with acute myeloid leukemia: correlation to cytogenetics, FAB subtype, and prognosis in theAMLCG study and usefulness as a marker for the detection of minimal residual disease. Blood, 1 Jul. 2002—volume 100, number 1.

Sorror M L1, Mans M B, Storb R, Baron F, Sandmaier B M, Maloney D G, Storer B. Hematopoietic cell transplantation (HCT)-specific comorbidity index: a new tool for risk assessment before allogeneic HCT. Blood. 2005 Oct. 15;106(8):2912-9. Epub 2005 Jun 30.

Sorror M L1, Sandmaier B M, Storer B E, Mans M B, Baron F, Maloney D G, Scott B L, Deeg H J, Appelbaum F R, Storb R. Comorbidity and disease status based risk stratification of outcomes among patients with acute myeloid leukemia or myelodysplasia receiving allogeneic hematopoietic cell transplantation. J Clin Oncol. 2007 Sep 20;25(27):4246-54. Epub 2007 Aug. 27.

Sweet Kendra & Jeffrey E. Lancet. Novel Therapeutics in Acute Myeloid Leukemia.

Curr Hematol Malig Rep. 2014 Mar. 16.

Wu, J., Smith, L., Plass, C., Huang. ChIP-chip comes of age for genome-wide functional analysis. Cancer Res. 2006 Jul. 15 ;66(14):6899-902.

1. An in vitro method for predicting the survival time of a subject suffering from acute myeloid leukemia (AML) comprising determining, in a biological sample from the subject, the epigenetic profile of H3K27.

2. An in vitro method according to claim 1 wherein the step of determining the epigenetic profile of H3K27 comprises:
i) determining in the biological sample obtained from the subject a histone methylation profile level of H3K27,
ii) comparing the histone methylation profile level of H3K27 at step i) with a predetermined reference value, and
iii) providing a good prognosis when the histone methylation profile level determined at step i) is higher than the predetermined reference value, or providing a bad prognosis when the histone methylation profile level determined at step i) is lower than the predetermined reference value.

3. An in vitro method according to claim I wherein the step of determining the epigenetic profile of H3K27 comprises:
i) determining in the biological sample obtained from the subject a histone tri-methylation profile level of H3K27,
ii) comparing the histone tri-methylation profile level of H3K27 at step i) with a predetermined reference value, and
iii) providing a good prognosis when the histone tri-methylation profile level determined at step i) is higher than the predetermined reference value, or providing a bad prognosis when the histone tri-methylation profile level determined at step i) is lower than the predetermined reference value.

4. An in vitro method for predicting the survival time of a subject suffering from acute myeloid leukemia (AML) comprising determining, in a biological sample from the subject, an epigenetic profile of H3K27 at an the HIST1 cluster located on 6p22.2.

5. An in vitro method for predicting the survival time of a subject suffering from acute myeloid leukemia (AML) as recited in claim 4 wherein the HIST1 cluster located on 6p22.2 is at position 26216000-2628500.

6. An in vitro method according to claim 1 wherein the acute myeloid leukemia (AML) is an acute myeloid leukemia with normal karyotype (CN-AML).

7.-8. (canceled)

9. An in vitro method for predicting the survival time of a subject suffering from acute myeloid leukemia (AML) comprising i) determining in a sample obtained from the subject a histone methylation profile level of H3K27 and the presence or absence of NPM1 mutations, ii) comparing the histone methylation profile level of H3K27 at step i) with a predetermined reference value, and iii) providing a good prognosis when the histone methylation profile level determined at step i) is higher than the predetermined reference value and when there is a mutation in NPM1, providing a good prognosis when the histone methylation profile level determined at step i) is higher than the predetermined reference value and when there is no mutation in NPM1 and providing a bad prognosis when the histone methylation profile level determined at step i) is lower than its predetermined reference value and when there is a mutation in NPM1 or where there is no mutation in NPM1.

10. A method of treatment of an AML in a subject in need thereof comprising the step of:
a) determining the epigenetic profile of the H3K27 according to claim 1 and;
b) administrating to said subject a compound useful for the treatment of AML when the prognosis of the subject is bad as determined by the method of the invention.

11. An in vitro method according to claim 4 wherein the acute myeloid leukemia (AML) is an acute myeloid leukemia with normal karyotype (CN-AML).
